(19) United States
(12) Patent Application Publication
(10) Pub. No.: US 2015/0025133 A1
(43) Pub. Date: Jan. 22, 2015

(54) NUTRITIONAL COMPOSITIONS INCLUDING RRR-ALPHA TOCOPHEROL AND POLYUNSATURATED FATTY ACIDS

(71) Applicant: ABBOTT LABORATORIES, ABBOTT PARK, IL (US)

(72) Inventors: Chron-Si Lai, Blacklick, OH (US); Matthew J. Kuchan, Westerville, OH (US); Daniel S. Albrecht, Columbus, OH (US)

(73) Assignee: ABBOTT LABORATORIES, ABBOTT PARK, IL (US)

(21) Appl. No.: 14/384,739

(22) PCT Filed: Mar. 7, 2013
(86) PCT No.: PCT/US13/29611

§ 371 Date: Sep. 12, 2014

Related U.S. Application Data
(60) Provisional application No. 61/610,799, filed on Mar. 14, 2012.

(51) Int. Cl. A61K 31/355; A23L 1/29; A61K 31/047; A61K 31/202; A61K 31/341

(52) U.S. Cl. CPC A61K 31/355; A61K 31/202; A61K 31/341; A61K 31/047; A23L 1/296; A23V 2002/00
USPC 514/458

(57) ABSTRACT

Disclosed are nutritional formulas generally, and infant formulas specifically, including a combination of RRR-alpha tocopherol, LC-PUFAs, and optionally vitamin C. The combination enhances brain development and improves cognitive performance in an individual, and specifically in an infant.

### CROSS REFERENCE TO RELATED APPLICATION

The present invention hereby claims the benefit of the provisional patent application Ser. No. 61/610,799, filed Mar. 14, 2012, the disclosure of which is hereby incorporated by reference in its entirety.

### FIELD OF THE DISCLOSURE

The present disclosure relates to nutritional compositions, and particularly to infant formulas, including RRR-alpha tocopherol. More particularly, the present disclosure relates to nutritional compositions including RRR-alpha tocopherol, long chain polyunsaturated fatty acids (LC-PUFAs), and vitamin C and to the methods of administering the compositions for enhancing brain development and/or improving cognitive performance in an individual, and specifically an infant.

### BACKGROUND OF THE DISCLOSURE

Infant formulas are commonly used today to provide a supplemental or sole source of nutrition early in life to both preterm and term infants. These formulas typically contain protein, carbohydrate, fat, vitamins, minerals, and other nutrients, and are commercially available as powders, ready-to-feed liquids, and liquid concentrates. Many infant formulas provide a quality alternative to human milk as not all infants can receive human milk.

In addition to the nutrients noted above, long chain polyunsaturated fatty acids (LC-PUFAs), including arachidonic acid (ARA) and docosahexaenoic acid (DHA), and tocopherols, including RRR-alpha tocopherol, are present in many infant formulas. ARA and DHA, along with other fatty acids, are generally believed to support brain and vision development in infants, as well as provide other benefits, while tocopherols may provide antioxidant benefits for stabilizing unsaturated lipids in cell membranes against autooxidation and scavenge free radicals produced by lipid peroxidation by the normal activity of oxidative enzymes.

LC-PUFAs included in infant formulas can be susceptible to damage by oxidation and degradation. In some cases, a high level of DHA and/or ARA may result in increased generation of lipid peroxides that can degrade RRR-alpha tocopherol before the RRR-alpha tocopherol can be absorbed by the gut. Additionally, xanthin oxidase (XO) reacts with AMP in the intestine to produce hydrogen peroxide, which may help an infant to prevent bacteria getting through the junction point. However, the newborn infant's anti-oxidation enzymes are not well developed. As a result, the hydrogen peroxide from XO may also oxidize lipids, such as LC-PUFAs, resulting in degradation of the RRR-alpha tocopherol. To combat this unwanted effect, one or more antioxidants can be included in the infant formula to provide some protection from oxidation and degradation of the LC-PUFAs.

There is a continuing need in the art for stable nutritional formulas, including infant formulas, that can provide a wide range of nutrients to an infant while maintaining stability over an extended period of time.

### SUMMARY OF THE DISCLOSURE

The present disclosure is directed to nutritional compositions, and infant formulas in particular, that include a combination of RRR-alpha tocopherol, DHA, ARA, and vitamin C. The compositions may improve the maturation of the central nervous system in an infant resulting from the stimulation of cholesterol production in the brain by the RRR-alpha tocopherol accretion and the resulting cholesterol-stimulated neuron myelination. In many embodiments, the nutritional compositions further include a protein, a fat, and a carbohydrate source. In some embodiments, methods for using the nutritional compositions include methods of improving the central nervous system maturation and cognition of an infant.

Some embodiments are directed to a nutritional composition comprising at least 7 mg/L of RRR-alpha tocopherol, at least 60 mg/L of docosahexaenoic acid, at least 110 mg/L of arachidonic acid, and at least 130 mg/L of vitamin C.

Some embodiments are directed to a method for enhancing brain development in an infant. The method comprises administering to an infant an infant formula comprising at least 7 mg/L of RRR-alpha tocopherol, at least 60 mg/L of docosahexaenoic acid, at least 110 mg/L of arachidonic acid, and at least 130 mg/L of vitamin C.

Some embodiments are directed to a method for improving cognitive performance in an infant. The method comprises administering to an infant an infant formula comprising at least 7 mg/L of RRR-alpha tocopherol, at least 60 mg/L of docosahexaenoic acid, at least 110 mg/L of arachidonic acid, and at least 130 mg/L of vitamin C.

Some embodiments are directed to a nutritional composition comprising at least 7 mg/L of RRR-alpha tocopherol, at least 60 mg/L of docosahexaenoic acid, and at least 110 mg/L of arachidonic acid.

It has now been found that nutritional compositions, such as infant formulas, including specific combinations and amounts of RRR-alpha tocopherol, DHA, and ARA provide improved central nervous system maturation and cognitive development, functioning and/or performance to an infant due to the enhance brain accretion of the RRR-alpha tocopherol. Further, the addition of vitamin C to the combination of RRR-alpha tocopherol, DHA, and ARA provides protection against oxidation of DHA and ARA, thereby further protecting RRR-alpha tocopherol from degradation and allowing RRR-alpha tocopherol, DHA, and ARA to be better absorbed by the gut of the individual.

Accordingly, the nutritional compositions and methods of the present disclosure offer an alternative therapeutic or nutritional intervention option that may contribute to the enhancement of brain development, enhanced central nervous system development, and improvement of cognitive performance, in individuals, and particularly in infants, toddlers, and children.

### DETAILED DESCRIPTION OF THE DISCLOSURE

The compositions and methods disclosed herein are directed to nutritional compositions generally, and infant formulas specifically, that include a combination of RRR-alpha tocopherol, DHA, ARA, and vitamin C. Through accretion of the RRR-alpha tocopherol in the brain, these compositions are capable of enhancing infant brain development, enhancing infant central nervous system development, and improving the general cognitive performance of an infant.

It is imperative that early in life infants receive sufficient nutrition to provide for adequate maturation both physically and mentally, and specifically in the brain and central nervous system. Insufficient nutrition can result in numerous health problems that can be life-long in many individuals. Brain and central nervous system maturation are key developmental areas for infants. Through brain accretion of the RRR-alpha tocopherol from the nutritional compositions described herein, the central nervous system maturation of an infant may be enhanced and improved through improved neuron myelination. Additionally, the brain accretion of the RRR-alpha tocopherol stimulates the brain glutamate synthesis, which enhances and stimulates neonatal neuron elongation and branching, which leads to the establishment of gap junctions between neurons. As such, use of the nutritional compositions as described herein provides the necessary nutrients to allow infants to develop mature central nervous systems and improved cognition.

These and other elements or features of the various embodiments are described in detail hereafter.

The terms “retort” and “retort sterilized” are used interchangeably herein, and unless otherwise specified, refer to the common practice of filling a container, most typically a metal can or other similar package, with a nutritional liquid, such as a liquid infant formula, and then subjecting the liquid-filled package to the necessary heat sterilization step, to form a retort sterilized nutritional liquid product.

The terms “aseptic” and “aseptic sterilized” are used interchangeably herein, and unless otherwise specified, refer to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product.

The terms “nutritional composition,” “nutritional product,” and “nutritional formula” as used herein, unless otherwise specified, are used interchangeably to refer to nutritional liquids and nutritional powders that comprise at least one of protein, fat, and carbohydrate and are suitable for oral administration to a human. The nutritional composition may further comprise vitamins, minerals, and other ingredients and represent a sole, primary, or supplemental source of nutrition. Nutritional compositions include infant formulas.

The term “nutritional liquid,” as used herein, unless otherwise specified, refers to nutritional products in ready-to-drink liquid form, concentrated form, and nutritional liquids made by reconstituting the nutritional powders described herein prior to use.

The term “nutritional powder,” as used herein, unless otherwise specified, refers to nutritional products in flowable or scoopable form that can be reconstituted with water or another aqueous liquid prior to consumption and includes both spray dried and drymixed/dryblended powders.

The terms “fat,” “lipid” and “oil” as used herein, unless otherwise specified, are used interchangeably to refer to lipid materials derived or processed from plants or animals. These terms also include synthetic lipid materials so long as such synthetic materials are suitable for oral administration to humans.

The term “cognitive performance” as used herein, unless otherwise specified, refers to the learning, thinking, and memory functions (i.e., memory acquisition, memory retention and memory recall) of the brain. Accordingly, the term “improving cognitive performance” as used herein, unless otherwise specified, refers to improving the learning, thinking, and/or memory (memory acquisition, memory retention and memory recall) functions of an infant.

All percentages, parts and ratios as used herein, are by weight of the total product, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the nutritional compositions of the present disclosure may also be substantially free of any ingredient or feature described herein, provided that the remaining formula still contains all of the required ingredients or features as described herein. In this context, and unless otherwise specified, the term “substantially free” means that the selected composition contains less than a functional amount of the optional ingredient, typically less than 1%, including less than 0.5%, including less than 0.1%, and also including zero percent, by weight of such optional or selected essential ingredient.

The nutritional compositions may comprise, consist of, or consist essentially of the elements of the products as described herein, as well as any additional or optional element described herein or otherwise useful in nutritional product applications.

### Product Form

The nutritional compositions of the present disclosure in some embodiments include a combination of RRR-alpha-tocopherol, LC-PUFAs, and vitamin C and may be formulated and administered in any known or otherwise suitable oral product form. Any solid, semi-solid, liquid, semi-liquid, or powder form, including combinations or variations thereof, are suitable for use herein, provided that such forms allow for safe and effective oral delivery to the individual of the essential ingredients as also defined herein.

Specific non-limiting examples of product forms suitable for use with products and methods disclosed herein include, for example, liquid and powder preterm infant formulas, liquid and powder term infant formulas, liquid and powder toddler formulas, and liquid and powder elemental and semi-elemental formulas. Adult nutritional formulas are also within the scope of the present disclosure.

The nutritional compositions of the present disclosure are preferably formulated as dietary product forms, which are defined herein as those embodiments comprising the ingredients of the present disclosure in a product form that also contains at least one of fat, protein, and carbohydrate. The compositions may be formulated with sufficient kinds and amounts of nutrients to provide a sole, primary, or supplemental source of nutrition, or to provide a specialized nutritional product such as for use in infants afflicted with specific diseases or conditions or with a targeted nutritional benefit.

### Nutritional Liquids

Nutritional liquids include both concentrated and ready-to-feed nutritional liquids. These nutritional liquids are most typically formulated as suspensions, emulsions or clear or substantially clear liquids.

Nutritional emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional liquids may be and typically are shelf stable. The nutritional liquids typically contain up to 95% by weight of water, including from about 50% to about 95%, also including from about 60% to about 90%, and also including from about 70% to about 85%, of water by weight of the nutritional liquid. The nutritional liquids may have a variety of product densities, but most typically have a density greater than 1.03 g/mL, including greater than 1.04 g/mL, including greater than 1.055 g/mL, including from about 1.06 g/mL to about 1.12 g/mL, and also including from about 1.085 g/mL to about 1.10 g/mL.

The nutritional liquid may have a pH ranging from about 3.5 to about 8, but are most advantageously in a range of from about 4.5 to about 7.5, including from about 5.5 to about 7.3, including from about 6.2 to about 7.2.

Although the serving size for the nutritional liquid can vary depending upon a number of variables, a typical serving size is generally at least 2 mL, or even at least 5 mL, or even at least 10 mL, or even at least 25 mL, including ranges from 2 mL to about 300 mL, including from about 100 mL to about 300 mL, from about 4 mL to about 250 mL, from about 150 mL to about 250 mL, from about 10 mL to about 240 mL, and from about 190 mL to about 240 mL.

### Nutritional Powders

The nutritional powders are in the form of flowable or substantially flowable particulate compositions, or at least particulate compositions. Particularly suitable nutritional powder forms include spray dried, agglomerated or dryblended powder compositions, or combinations thereof, or powders prepared by other suitable methods. The compositions can easily be scooped and measured with a spoon or similar other device, wherein the compositions can easily be reconstituted with a suitable aqueous liquid, typically water, to form a nutritional liquid, such as an infant formula, for immediate oral or enteral use. In this context, “immediate” use generally means within about 48 hours, most typically within about 24 hours, preferably right after or within 20 minutes of reconstitution.

### RRR-Alpha Tocopherol

The nutritional compositions of the present disclosure include RRR-alpha tocopherol. As used herein, the term “RRR-alpha tocopherol” refers to both exogenous sources and inherent sources of RRR-alpha tocopherol and RRR-alpha tocopherol acetate that are present in a nutritional composition, including an infant formula. Inherent sources include RRR-alpha tocopherol that is inherently present in components that are present in a nutritional composition and may include for example, various oils and fats. Exogenous sources of RRR-alpha tocopherol include RRR-alpha tocopherol that is added to the nutritional composition not as part of another component.

It has been discovered that brain accretion of RRR-alpha tocopherol enhances the central nervous system maturation and cognition; that is, the presence of RRR-alpha tocopherol in the brain of a human infant enhances the maturation of the infant's central nervous system and cognitive development. The presence of elevated levels of RRR-alpha tocopherol in the brain may increase the production of cholesterol in the brain, which leads to increased neuron myelination. Also, the brain accretion of RRR-alpha tocopherol stimulates the production of glutamate in the brain, which can result in neuron elongation and branching, which can lead to the establishment of gap junctions between neurons. This gap communication can significantly increase the communication speed between neurons and allow the brain to process more data in a shorter time.

Tocopherols, generically referred to as vitamin E, are available in four forms, alpha, beta, gamma, and delta, which differ in the number and position of the methyl groups on the chroman ring (see Table 1). Further, tocopherols can exist in a number of stereoisomeric forms depending on the chirality of the phytyl tail. Of the alpha tocopherols, RRR-alpha tocopherol (also referred to as “natural vitamin E”) has the greatest biological activity and is reported to be the dominant form of the alpha tocopherol in the brain. RRR-alpha tocopherol is a single stereoisomer whereas synthetic vitamin E (all-rac-alpha tocopherol or tocopherol acetate) is an equimolar mixture of eight isomers, only one of which is RRR-alpha tocopherol. The fact that the dominant form of alpha tocopherol is RRR alpha tocopherol (based on animal studies) strongly suggests that the other seven chiral isomers must be absorbed at a lower rate by the brain or oxidized at a faster rate. Cholesterol is a major component of myelin, and it is likely that stimulated cholesterol synthesis may stimulate newborn infant neuron myelination. Glutamate has been shown to stimulate neurite outgrowth and branching; neurite outgrowth and branching allows neuron cells to establish gap junctions with multiple neurons; the discovery that RRR alpha tocopherol correlates with glutamate and cholesterol suggests that RRR alpha tocopherol plays a key role in newborn infant central nervous system maturation.

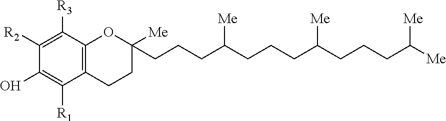

TABLE 1

| Compound | R₁ | R₂ | R₃ |
|---|---|---|---|
| alpha-tocopherol | Me | Me | Me |
| beta-tocopherol | Me | H | Me |
| gamma-tocopherol | H | Me | Me |
| delta-tocopherol | H | H | Me |

The RRR-alpha tocopherol is present in the nutritional compositions in concentrations of at least 7 mg/L, including at least 8 mg/L, including at least 9 mg/L, including at least 10 mg/L, including at least 15 mg/L, including at least 18 mg/L, including at least 20 mg/L, including from at least 7 mg/L to about 100 mg/L, including from at least 7 mg/L to about 50 mg/L, and including from about 20 mg/L to about 40 mg/L of the infant formula. The total amounts of RRR-alpha tocopherol include both exogenous and inherent amounts of RRR-alpha tocopherol, as noted above.

In some embodiments, the nutritional compositions include another additional tocopherol, particularly gamma-tocopherol, in addition to the RRR-alpha tocopherol. Gamma tocopherol has been used in food applications as an antioxidant, thereby preventing deterioration of foods and beverages resulting from oxidation of susceptible components such as some fats.

Gamma tocopherol, however, has now been found to negatively correlate with phospholipids. Accordingly, when present, the gamma tocopherol is present in the infant formulas in concentrations of less than 7 mg/L, including less than 5 mg/L, including from 0 mg/L to 3 mg/L. including from about 1 mg/L to 3 mg/L of the nutritional composition.

### Long Chain Polyunsaturated Fatty Acids (LC-PUFAs)

The nutritional compositions of the present disclosure include LC-PUFAs in addition to the RRR-alpha tocopherol. LC-PUFAs are included in the nutritional compositions to provide nutritional support and benefits, as well as to support brain development in individuals, and specifically in infants. In some embodiments, the nutritional compositions include a combination of LC-PUFAs with the RRR-alpha tocopherol. Particularly suitable for use in the nutritional compositions in addition to the RRR-alpha tocopherol, are combinations of arachidonic acid (ARA) and docosahexaenoic acid (DHA).

DHA is an n-3 LC-PUFA and is abundant in the brain and retina, accounting for 40% of the LC-PUFAs in the brain and 60% of the LC-PUFAs in the retina. ARA is an n-6 LC-PUFA that is present in the phospholipids, especially phosphatidylethanolamine, phosphatidylcholine, and phosphatidylinositides, of membranes of the body's cells, and is abundant in the brain, muscles, and liver.

The LC-PUFAs may be provided as free fatty acids, in triglyceride form, in diglyceride form, in monoglyceride form, in phospholipid form, or as a mixture of one or more of the above, preferably in triglyceride form.

In some embodiments, the nutritional compositions include DHA in a concentration of at least 60 mg/L, including at least 70 mg/L, including at least 80 mg/L, including at least 90 mg/L, including at least 100 mg/L, including at least 150 mg/L, including at least 200 mg/L and including from 60 mg/L to about 1000 mg/L, and including from about 100 mg/L to about 500 mg/L, and include ARA in a concentration of at least 110 mg/L, including at least 120 mg/L, including at least 130 mg/L, including at least 140 mg/L, including at least 150 mg/L, including at least 200 mg/L, and including from 110 mg/L to about 1000 mg/L, and also including from about 110 mg/L to about 500 mg/L.

In some embodiments, the nutritional compositions include combinations of RRR-alpha tocopherol, DHA and ARA such that the weight ratio of DHA to RRR-alpha tocopherol ranges from about 5:1 to about 15:1, desirably from about 7.5:1 to about 10:1, and the weight ratio of ARA to RRR-alpha tocopherol ranges from about 12:1 to about 24:1, desirably from about 12:1 to about 18:1.

### Vitamin C

The nutritional compositions of the present disclosure further include vitamin C in addition to the RRR-alpha tocopherol and LC-PUFA to provide oxidative protection. Vitamin C, also referred to as L-ascorbic acid or L-ascorbate, is available from many fruit and vegetable sources. Any source of vitamin C that is suitable for use in an oral nutritional product and is compatible with the essential elements and features of such products may be used with the nutritional compositions of the present disclosure.

It has recently been found that vitamin C may chelate free ferrous iron, which has been found to lower serum vitamin E levels in formula fed pre-term infants, thereby preventing iron from acting as a pro-oxidant. Further, high levels of ARA and DHA may generate high levels of lipid peroxides due to oxidation induced by intestine xanthin oxidase (XO), which can also degrade RRR-alpha tocopherol before RRR-alpha tocopherol can be absorbed in the gut. Accordingly, including vitamin C in the infant formulas of the present disclosure may reduce the oxidative degradation of RRR-alpha tocopherol.

The nutritional compositions of the present disclosure desirably include vitamin C in a concentration of at least 130 mg/L, including at least 150 mg/L, including at least 175 mg/L, including at least 200 mg/L, including at least 225 mg/L, including at least 250 mg/L, including at least 300 mg/L and including from 130 mg/L to about 1000 mg/L, and including from about 200 mg/L to about 500 mg/L.

### Carotenoids

In some embodiments, the nutritional compositions additionally include carotenoids to provide additional oxidative protection, as well as to further enhance brain development of the infant. In exemplary embodiments, the nutritional compositions include lutein, beta-carotene, zeaxanthin, lycopene, and combinations thereof. In some embodiments, the nutritional composition includes one or more of lutein and zeaxanthin.

It is generally desirable that the nutritional composition comprises at least one of lutein, lycopene, zeaxanthin, beta-carotene to provide a total amount of carotenoid of from about 0.001 μg/mL to about 5 μg/mL. More particularly, the nutritional compositions comprise lutein in an amount of from 0.001 μg/mL to 5 μg/mL, including from 0.001 μg/mL to 0.0190 μg/mL, including from 0.001 μg/mL to 0.0140 μg/L, and also including from 0.044 μg/mL to 5 μg/mL of lutein. It is also generally desirable that the nutritional compositions comprise from 0.001 μg/mL to 5 μg/mL, from 0.001 μg/mL to 0.0130 μg/mL, including from 0.001 μg/mL to 0.0075 μg/mL of lycopene, and also including from 0.0185 μg/L to 5 μg/L of lycopene. It is also generally desirable that the nutritional compositions comprise from 1 μg/mL to 5 μg/mL, including from 0.001 μg/mL to 0.025 μg/L of beta-carotene, including from 0.001 μg/L to 0.011 μg/mL of beta-carotene, and also including from 0.034 μg/mL to 5 μg/mL of beta-carotene. It should be understood that any combination of these amounts of beta-carotene, lutein, zeaxanthin, and lycopene can be included in the nutritional compositions of the present disclosure. Other carotenoids may optionally be included in the infant formulas as described herein. Any one or all of the carotenoids included in the infant formulas described herein may be from a natural source, or artificially synthesized.

Each of the carotenoids in the selected combinations can be obtained from any known or otherwise suitable material source for use in infant formulas, and each can be provided individually, or all together, or in any combination and from any number of sources, including sources such as multivitamin premixes containing other vitamins or minerals in combination with one or more of the carotenoids as described herein. Non-limiting examples of some suitable sources of lutein, lycopene, beta-carotene, or combinations thereof include LycoVit® lycopene (available from BASF, Mount Olive, N.J.), Lyc-O-Mato® tomato extract in oil, powder, or bead form (available from LycoRed Corp., Orange, N.J.), beta-carotene, lutein, or lycopene (available from DSM Nutritional Products, Parsippany, N.J.), FloraGLO® lutein (available from Kemin Health, Des Moines, Iowa), Xangold® Natural Lutein Esters (available from Cognis, Cincinnati, Ohio), and Lucarotin® beta-carotene (available from BASF, Mount Olive, N.J.).

### Macronutrients

The nutritional compositions of the present disclosure may further comprise one or more optional macronutrients in addition to the RRR-alpha tocopherol, LC-PUFA, and vitamin C described herein. The optional macronutrients include proteins, lipids (in addition to the LC-PUFA), carbohydrates, and combinations thereof. The nutritional compositions are desirably formulated as dietary products containing all three macronutrients.

Macronutrients suitable for use herein include any protein, lipid (in addition to the LC-PUFA), or carbohydrate or source thereof that is known for or otherwise suitable for use in an oral nutritional composition, provided that the optional macronutrient is safe and effective for oral administration and is otherwise compatible with the other ingredients in the nutritional composition.

The concentration or amount of optional lipid (inclusive of the LC-PUFA), carbohydrate, and protein in the nutritional compositions can vary considerably depending upon the particular product form (e.g., bars or other solid dosage forms, milk or soy-based liquids or other clear beverages, reconstitutable powders, etc.) and the various other formulations and targeted dietary needs. These optional macronutrients are most typically formulated within any of the embodied ranges described in the following tables.

| Nutrient % Total Cal. | Embodiment A | Embodiment B | Embodiment C |
|---|---|---|---|
| Carbohydrate | 0-98 | 2-96 | 10-75 |
| Protein | 0-98 | 2-96 | 5-70 |
| Lipid | 0-98 | 2-96 | 20-85 |
| | Embodiment D | Embodiment E | Embodiment F |
| Carbohydrate | 30-50 | 25-50 | 25-50 |
| Protein | 15-35 | 10-30 | 5-30 |
| Lipid | 35-55 | 1-20 | 2-20 |

Each numerical value preceded by the term “about”

### Carbohydrate

Optional carbohydrates suitable for use in the nutritional compositions may be simple, complex, or variations or combinations thereof. Non-limiting examples of suitable carbohydrates include hydrolyzed or modified starch or cornstarch, maltodextrin, isomaltulose, sucromalt, glucose polymers, sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

Optional carbohydrates suitable for use herein also include soluble dietary fiber, non-limiting examples of which include gum Arabic, fructooligosaccharides (FOS), sodium carboxymethyl cellulose, guar gum, citrus pectin, low and high methoxy pectin, oat and barley glucans, carrageenan, psyllium and combinations thereof. Insoluble dietary fiber is also suitable as a carbohydrate source herein, non-limiting examples of which include oat hull fiber, pea hull fiber, soy hull fiber, soy cotyledon fiber, sugar beet fiber, cellulose, corn bran, and combinations thereof.

### Protein

Optional proteins suitable for use in the nutritional compositions include hydrolyzed, partially hydrolyzed or non-hydrolyzed proteins or protein sources, and can be derived from any known or otherwise suitable source such as milk (e.g., casein, whey), animal (e.g., meat, fish, egg albumen), cereal (e.g., rice, corn), vegetable (e.g., soy, pea, potato), or combinations thereof. The proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

### Lipid

Optional lipids suitable for use in the nutritional compositions, which are optionally in addition to the LC-PUFAs as described herein, include coconut oil, fractionated coconut oil, soy oil, corn oil, olive oil, safflower oil, high oleic safflower oil, high GLA-safflower oil, MCT oil (medium chain triglycerides), sunflower oil, high oleic sunflower oil, palm and palm kernel oils, palm olein, canola oil, flaxseed oil, borage oil, cottonseed oils, evening primrose oil, blackcurrant seed oil, transgenic oil sources, fungal oils, marine oils (e.g., tuna, sardine), and so forth.

### Optional Ingredients

The nutritional compositions may further comprise other optional ingredients that may modify the physical, nutritional, chemical, hedonic or processing characteristics of the formulas or serve as pharmaceutical or additional nutritional components when used in a targeted population. Many such optional ingredients are known or otherwise suitable for use in other nutritional products and may also be used in the nutritional compositions described herein, provided that such optional ingredients are safe and effective for oral administration and are compatible with the essential and other ingredients in the composition.

Non-limiting examples of such other optional ingredients include preservatives, anti-oxidants, buffers, pharmaceutical actives, sweeteners, colorants, flavors, flavor enhancers, thickening agents and stabilizers, emulsifying agents, lubricants, and combinations thereof.

The nutritional compositions may further include one or more minerals, non-limiting examples of which include phosphorus, sodium, chloride, magnesium, manganese, iron, copper, zinc, iodine, calcium, potassium, chromium, molybdenum, selenium, and combinations thereof.

The nutritional compositions may also include one or more vitamins (in addition to vitamin C), non-limiting examples of which include biotin, choline, inositol, folic acid, pantothenic acid, TPAN, choline, vitamin A, thiamine (vitamin B1), riboflavin (vitamin B2) niacin (vitamin B3), pyridoxine (vitamin B6), cyanocobalamin (vitamin B12), vitamin D, vitamin E, vitamin K, and various salts, esters, or other derivatives thereof, and combinations thereof.

### Methods of Manufacture

The nutritional compositions may be prepared by any known or otherwise effective manufacturing technique for preparing the selected product form. Many such techniques are known for any given product form such as nutritional liquids and nutritional powders and can easily be applied by one of ordinary skill in the nutrition and formulation arts to the nutritional products described herein.

Liquid, milk or soy-based nutritional liquids, for example, may be prepared by first forming an oil and fiber blend containing all formulation oils, any emulsifier, fiber and fat-soluble vitamins. Additional slurries (typically a carbohydrate and two protein slurries) are prepared separately by mixing the carbohydrate and minerals together and the protein in water. The slurries are then mixed together with the oil blend. The resulting mixture is homogenized, heat processed, standardized with any water-soluble vitamins, flavored and the liquid terminally sterilized or aseptically filled or dried to produce a powder.

The nutritional compositions of the present disclosure may also be manufactured by other known or otherwise suitable techniques not specifically described herein without departing from the spirit and scope of the present disclosure. The present embodiments are, therefore, to be considered in all respects as illustrative and not restrictive and that all changes and equivalents also come within the description of the present disclosure.

### Methods of Use

The methods of the present disclosure include the oral administration of the nutritional compositions, and specifically infant formulas, that include RRR-alpha tocopherol in combination with LC-PUFA (particularly DHA and ARA), and vitamin C, to enhance brain development. As RRR-alpha tocopherol, DHA, and ARA are each highly concentrated in the brain, the inclusion of RRR-alpha tocopherol in combination with LC-PUFAs such as DHA and ARA as described herein, provides for critical nutrients (e.g., cholesterol and phospholipids) needed for brain development in individuals, and specifically infants.

In addition to enhancing brain development, the nutritional compositions can be administered to improve cognitive performance in an infant. Particularly, the combination of RRR-alpha tocopherol, DHA, ARA, and vitamin C may improve general cognition by enhancing memory acquisition, memory retention and memory recall that contributes to the cognitive functions of learning, thinking, and memory.

The nutritional compositions as described herein can be administered to individuals including infants generally, or may, in some embodiments, be administered to a specific subclass of infants that are “in need thereof;” that is, to specific infants that would specifically benefit by administration of the infant formula. For example, a specific infant may be “in need of” the infant formulas as described herein if they are susceptible to (i.e., genetically predisposed, have a family history of, and/or having symptoms of the disease or condition) neurodegenerative diseases or other diseases and conditions that can impair/reduce cognition generally or specific aspects of cognition.

The individual desirably consumes at least one serving of the nutritional composition daily, and in some embodiments, may consume two, three, or even more servings per day. Each serving is desirably administered as a single, undivided dose, although the serving may also be divided into two or more partial or divided servings to be taken at two or more times during the day. The methods of the present disclosure include continuous day after day administration, as well as periodic or limited administration, although continuous day after day administration is generally desirable. The methods of the present disclosure are preferably applied on a daily basis, wherein the daily administration is maintained continuously for at least 3 days, including at least 5 days, including at least 1 month, including at least 6 weeks, including at least 8 weeks, including at least 2 months, including at least 6 months, desirably for at least about 18-24 months, desirably as a long term, continuous, daily, dietary source or supplement.

### EXAMPLES

The following examples illustrate specific embodiments and or features of the nutritional products of the present disclosure. The examples are given solely for the purpose of illustration and are not to be construed as limitations of the present disclosure, as many variations thereof are possible without departing from the spirit and scope of the disclosure.

The exemplified products are nutritional products prepared in accordance with manufacturing methods well known in the nutrition industry for preparing nutritional liquids (e.g., emulsions) and powders.

### Examples 1-5

Examples 1-5 illustrate ready-to-feed nutritional emulsions of the present disclosure, the ingredients of which are listed in the table below. All ingredient amounts are listed as kilogram per 1000 kilogram batch of product, unless otherwise specified.

| Ingredient | Ex. 1 | | Ex. 2 | | Ex. 3 | | Ex. 4 | | Ex. 5 | |
|---|---|---|---|---|---|---|---|---|---|---|
| Water | Q.S. | | Q.S. | | Q.S. | | Q.S. | | Q.S. | |
| Condensed Skim Milk | 86.64 | | 86.64 | | 86.64 | | 86.64 | | 86.64 | |
| Lactose | 54.80 | | 54.80 | | 54.80 | | 54.80 | | 54.80 | |
| High oleic safflower oil | 14.10 | | 14.10 | | 14.10 | | 14.10 | | 14.10 | |
| Soybean oil | 10.6 | | 10.6 | | 10.6 | | 10.6 | | 10.6 | |
| Coconut oil | 10.1 | | 10.1 | | 10.1 | | 10.1 | | 10.1 | |
| 3′ sialylallactose | 0.0948 | | 0.090 | | 0.085 | | 9.479 | | 9.005 | |
| Galactooligosaccharides | 8.63 | | 8.63 | | 8.63 | | 8.63 | | 8.63 | |
| Whey protein concentrate | 6.40 | | 6.40 | | 6.40 | | 6.40 | | 6.40 | |
| Potassium citrate | 478.9 | g | 478.9 | g | 478.9 | g | 478.9 | g | 478.9 | g |
| Calcium carbonate | 448.28 | g | 448.28 | g | 448.28 | g | 448.28 | g | 448.28 | g |
| Soy lecithin | 355.74 | g | 355.74 | g | 355.74 | g | 355.74 | g | 355.74 | g |
| Stabilizer | 355.74 | g | 355.74 | g | 355.74 | g | 355.74 | g | 355.74 | g |
| ARA oil | 110 | g | 150 | g | 200 | g | 250 | g | 300 | g |
| Nucleotide/chloride premix | 293.26 | g | 293.26 | g | 293.26 | g | 293.26 | g | 293.26 | g |
| Potassium chloride | 226.45 | g | 226.45 | g | 226.45 | g | 226.45 | g | 226.45 | g |
| Ascorbic acid | 445.94 | g | 445.94 | g | 445.94 | g | 445.94 | g | 445.94 | g |
| Vitamin mineral premix | 142.88 | g | 142.88 | g | 142.88 | g | 142.88 | g | 142.88 | g |
| Vitamin C | 130 | g | 200 | g | 250 | g | 300 | g | 350 | g |
| DHA oil | 60 | g | 100 | g | 120 | g | 150 | g | 200 | g |
| Carrageenan | 180.0 | g | 180.0 | g | 180.0 | g | 180.0 | g | 180.0 | g |
| Magnesium chloride | 55.0 | g | 55.0 | g | 55.0 | g | 55.0 | g | 55.0 | g |
| Ferrous sulfate | 58.0 | g | 58.0 | g | 58.0 | g | 58.0 | g | 58.0 | g |
| Choline chloride | 53.9 | g | 53.9 | g | 53.9 | g | 53.9 | g | 53.9 | g |
| Vitamin ADEK premix | 47.4 | g | 47.4 | g | 47.4 | g | 47.4 | g | 47.4 | g |
| RRR-alpha tocopherol acetate | 7 | g | 20 | g | 30 | g | 40 | g | 50 | g |
| Citric acid | 29.77 | g | 29.77 | g | 29.77 | g | 29.77 | g | 29.77 | g |
| Mixed carotenoid premix | 26.40 | g | 26.40 | g | 26.40 | g | 26.40 | g | 26.40 | g |
| Sodium chloride | AN | | AN | | AN | | AN | | AN | |
| L-carnitine | 3.31 | g | 3.31 | g | 3.31 | g | 3.31 | g | 3.31 | g |
| Tricalcium phosphate | 15.65 | g | 15.65 | g | 15.65 | g | 15.65 | g | 15.65 | g |
| Potassium phosphate monobasic | 13.67 | g | 13.67 | g | 13.67 | g | 13.67 | g | 13.67 | g |
| Riboflavin | 2.42 | g | 2.42 | g | 2.42 | g | 2.42 | g | 2.42 | g |

| | | | | | |
|---|---|---|---|---|---|
| Potassium hydroxide | AN | AN | AN | AN | AN |

AN = as needed

### Examples 6-10

Examples 6-10 illustrate ready-to-feed nutritional emulsions of the present disclosure, the ingredients of which are listed in the table below. All ingredient amounts are listed as kilogram per 1000 kilogram batch of product, unless otherwise specified.

| Ingredient | Ex. 6 | | Ex. 7 | | Ex. 8 | | Ex. 9 | | Ex. 10 | |
|---|---|---|---|---|---|---|---|---|---|---|
| Water | Q.S. | | Q.S. | | Q.S. | | Q.S. | | Q.S. | |
| Condensed Skim Milk | 86.64 | | 86.64 | | 86.64 | | 86.64 | | 86.64 | |
| Lactose | 54.80 | | 54.80 | | 54.80 | | 54.80 | | 54.80 | |
| High oleic safflower oil | 14.10 | | 14.10 | | 14.10 | | 14.10 | | 14.10 | |
| Soybean oil | 10.6 | | 10.6 | | 10.6 | | 10.6 | | 10.6 | |
| Coconut oil | 10.1 | | 10.1 | | 10.1 | | 10.1 | | 10.1 | |
| 6′ sialylallactose | 0.0948 | | 0.0901 | | 0.0853 | | 9.479 | | 9.0047 | |
| Galactooligosaccharides | 8.63 | | 8.63 | | 8.63 | | 8.63 | | 8.63 | |
| Whey protein concentrate | 6.40 | | 6.40 | | 6.40 | | 6.40 | | 6.40 | |
| Potassium citrate | 478.9 | g | 478.9 | g | 478.9 | g | 478.9 | g | 478.9 | g |
| Calcium carbonate | 448.28 | g | 448.28 | g | 448.28 | g | 448.28 | g | 448.28 | g |
| Soy lecithin | 355.74 | g | 355.74 | g | 355.74 | g | 355.74 | g | 355.74 | g |
| Stabilizer | 355.74 | g | 355.74 | g | 355.74 | g | 355.74 | g | 355.74 | g |
| ARA | 110 | g | 150 | g | 200 | g | 250 | g | 300 | g |
| Nucleotide/chloride premix | 293.26 | g | 293.26 | g | 293.26 | g | 293.26 | g | 293.26 | g |
| Potassium chloride | 226.45 | g | 226.45 | g | 226.45 | g | 226.45 | g | 226.45 | g |
| Ascorbic acid | 445.94 | g | 445.94 | g | 445.94 | g | 445.94 | g | 445.94 | g |
| Vitamin mineral premix | 142.88 | g | 142.88 | g | 142.88 | g | 142.88 | g | 142.88 | g |
| Vitamin C | 130 | g | 200 | g | 250 | g | 300 | g | 350 | g |
| DHA | 60 | g | 100 | g | 120 | g | 150 | g | 200 | g |
| Carrageenan | 180.0 | g | 180.0 | g | 180.0 | g | 180.0 | g | 180.0 | g |
| Magnesium chloride | 55.0 | g | 55.0 | g | 55.0 | g | 55.0 | g | 55.0 | g |
| Ferrous sulfate | 58.0 | g | 58.0 | g | 58.0 | g | 58.0 | g | 58.0 | g |
| Choline chloride | 53.9 | g | 53.9 | g | 53.9 | g | 53.9 | g | 53.9 | g |
| Vitamin ADEK premix | 47.40 | g | 47.40 | g | 47.40 | g | 47.40 | g | 47.40 | g |
| RRR-alpha tocopherol acetate | 7 | g | 20 | g | 30 | g | 40 | g | 50 | g |
| Citric acid | 29.77 | g | 29.77 | g | 29.77 | g | 29.77 | g | 29.77 | g |
| Mixed carotenoid premix | 26.40 | g | 26.40 | g | 26.40 | g | 26.40 | g | 26.40 | g |
| Sodium chloride | AN | | AN | | AN | | AN | | AN | |
| L-carnitine | 3.31 | g | 3.31 | g | 3.31 | g | 3.31 | g | 3.31 | g |
| Tricalcium phosphate | 15.65 | g | 15.65 | g | 15.65 | g | 15.65 | g | 15.65 | g |
| Potassium phosphate monobasic | 13.67 | g | 13.67 | g | 13.67 | g | 13.67 | g | 13.67 | g |
| Riboflavin | 2.42 | g | 2.42 | g | 2.42 | g | 2.42 | g | 2.42 | g |

| | | | | | |
|---|---|---|---|---|---|
| Potassium hydroxide | AN | AN | AN | AN | AN |

AN = as needed

### Examples 11-15

Examples 11-15 illustrate spray dried nutritional powders of the present disclosure, the ingredients of which are listed in the table below. All ingredient amounts are listed as kilogram per 1000 kilogram batch of product, unless otherwise specified.

| Ingredient | Ex. 11 | | Ex. 12 | | Ex. 13 | | Ex. 14 | | Ex. 15 | |
|---|---|---|---|---|---|---|---|---|---|---|
| Condensed Skim Milk | 698.5 | | 698.5 | | 698.5 | | 698.5 | | 698.5 | |
| Lactose | 386.0 | | 386.0 | | 386.0 | | 386.0 | | 386.0 | |
| High oleic safflower oil | 114.4 | | 114.4 | | 114.4 | | 114.4 | | 114.4 | |
| Soybean oil | 85.51 | | 85.51 | | 85.51 | | 85.51 | | 85.51 | |
| Coconut oil | 78.76 | | 78.76 | | 78.76 | | 78.76 | | 78.76 | |
| 3′ sialylallactose | 0.3792 | | 0.3604 | | 0.3412 | | 37.916 | | 36.0188 | |
| Galactooligosaccharides | 69.50 | | 69.50 | | 69.50 | | 69.50 | | 69.50 | |
| Whey protein concentrate | 51.08 | | 51.08 | | 51.08 | | 51.08 | | 51.08 | |
| Potassium citrate | 9.168 | | 9.168 | | 9.168 | | 9.168 | | 9.168 | |
| Calcium carbonate | 4.054 | | 4.054 | | 4.054 | | 4.054 | | 4.054 | |
| Soy lecithin | 1.120 | | 1.120 | | 1.120 | | 1.120 | | 1.120 | |
| ARA | 825 | g | 1125 | g | 1500 | g | 1875 | g | 2250 | g |
| Nucleotide/chloride premix | 2.347 | | 2.347 | | 2.347 | | 2.347 | | 2.347 | |
| Potassium chloride | 1.295 | | 1.295 | | 1.295 | | 1.295 | | 1.295 | |
| Ascorbic acid | 1.275 | | 1.275 | | 1.275 | | 1.275 | | 1.275 | |
| Vitamin mineral premix | 1.116 | | 1.116 | | 1.116 | | 1.116 | | 1.116 | |
| Vitamin C | 975 | g | 1500 | g | 1875 | g | 2250 | g | 2625 | g |
| DHA | 60 | g | 100 | g | 120 | g | 150 | g | 200 | g |
| Magnesium chloride | 1.038 | | 1.038 | | 1.038 | | 1.038 | | 1.038 | |
| Sodium chloride | 579.4 | g | 579.4 | g | 579.4 | g | 579.4 | g | 579.4 | g |
| Ferrous sulfate | 453.6 | g | 453.6 | g | 453.6 | g | 453.6 | g | 453.6 | g |
| Choline chloride | 432.1 | g | 432.1 | g | 432.1 | g | 432.1 | g | 432.1 | g |
| Vitamin ADEK premix | 377.2 | g | 377.2 | g | 377.2 | g | 377.2 | g | 377.2 | g |
| RRR-alpha tocopherol acetate | 52.5 | g | 150 | g | 225 | g | 300 | g | 375 | g |
| Ascorbyl Palmitate | 361.3 | g | 361.3 | g | 361.3 | g | 361.3 | g | 361.3 | g |
| Mixed carotenoid premix | 350.1 | g | 350.1 | g | 350.1 | g | 350.1 | g | 350.1 | g |
| Mixed Tocopherols | 159.2 | g | 159.2 | g | 159.2 | g | 159.2 | g | 159.2 | g |
| L-carnitine | 26.30 | g | 26.30 | g | 26.30 | g | 26.30 | g | 26.30 | g |
| Riboflavin | 3.181 | g | 3.181 | g | 3.181 | g | 3.181 | g | 3.181 | g |

| | | | | | |
|---|---|---|---|---|---|
| Tricalcium phosphate | 0-5.23 | 0-5.23 | 0-5.23 | 0-5.23 | 0-5.23 |
| Potassium phosphate monobasic | 0-5.23 | 0-5.23 | 0-5.23 | 0-5.23 | 0-5.23 |
| Potassium hydroxide | AN | AN | AN | AN | AN |

AN = as needed

1-15. (canceled)

16. A nutritional composition comprising at least 7 mg/L of RRR-alpha tocopherol, at least 60 mg/L of docosahexaenoic acid, and at least 110 mg/L of arachidonic acid.

17. The nutritional composition of claim 16, further comprising at least 130 mg/L of vitamin C.

18. The nutritional composition of claim 16, further comprising less than 7 mg/L of gamma tocopherol.

19. The nutritional composition of claim 16, further comprising a carotenoid.

20. The nutritional composition of claim 19, wherein the carotenoid is selected from the group of lutein, zeaxanthin, and combinations thereof.

21. The nutritional composition of claim 16, wherein the weight ratio of docosahexaenoic acid to RRR-alpha tocopherol in the nutritional composition is from 5:1 to 15:1.

22. The nutritional composition of claim 16, wherein the weight ratio of arachidonic acid to RRR-alpha tocopherol in the nutritional composition is from 12:1 to 24:1.

23. The nutritional composition of claim 16, wherein the nutritional composition is an infant formula.

24. A method for enhancing brain development in an infant, the method comprising administering to an infant an infant formula comprising at least 7 mg/L of RRR-alpha tocopherol, at least 60 mg/L of docosahexaenoic acid, at least 110 mg/L of arachidonic acid, and at least 130 mg/L of vitamin C.

25. The method according to claim 24, wherein the weight ratio of docosahexaenoic acid to RRR-alpha tocopherol in the infant formula is from 5:1 to 15:1.

26. The method according to claim 24, wherein the weight ratio of arachidonic acid to RRR-alpha tocopherol in the infant formula is from 12:1 to 24:1.

27. The method according to claim 24, wherein the infant formula comprises less than 7 mg/L of gamma tocopherol.

28. The method according to claim 24, wherein the infant formula further comprises a carotenoid.

29. A method for improving cognitive performance in an infant, the method comprising administering to an infant an infant formula comprising at least 7 mg/L of RRR-alpha tocopherol, at least 60 mg/L of docosahexaenoic acid, at least 110 mg/L of arachidonic acid, and at least 130 mg/L of vitamin C.

30. The method according to claim 29, wherein the weight ratio of docosahexaenoic acid to RRR-alpha tocopherol in the infant formula is from 5:1 to 15:1.

31. The method according to claim 29, wherein the weight ratio of arachidonic acid to RRR-alpha tocopherol in the infant formula is from 12:1 to 24:1.

32. The method according to claim 29, wherein the infant formula comprises less than 7 mg/L of gamma tocopherol.

33. The method according to claim 29, wherein the infant formula further comprises a carotenoid.
